(19) United States
(12) Patent Application Publication
(10) Pub. No.: US 2017/0095368 A1
(43) Pub. Date: Apr. 6, 2017

(54) Device and Method for Heating a Predetermined Surface of a Human Body

(71) Applicant: Tommy M. Hess, Joliet, IL (US)

(72) Inventor: Tommy M. Hess, Joliet, IL (US)

(21) Appl. No.: 15/283,585

(22) Filed: Oct. 3, 2016

Related U.S. Application Data
(60) Provisional application No. 62/284,545, filed on Oct. 2, 2015.

(51) Int. Cl. A61F 7/02

(52) U.S. Cl. CPC A61F 7/02; A61F 2007/0035

(57) ABSTRACT

A device for heating a predetermined surface area of a human body, including but not limited to a person's wrist, neck and back, in accordance with the present invention includes a pouch for heating a predetermined surface area of a person's body; at least one heating element specified to heat the predetermined surface area of the person's body to a predetermined temperature; and a band or strap for securing the pouch to the predetermined surface area of the person's body, whereby, the pouch having at least one heating element inserted therein is secured upon the predetermined surface area of the person's body to ultimately heat the predetermined surface area to a predetermined temperature.

This Utility Application is based on Provisional Application No. 62/284,545, filed on Oct. 2, 2015.

### BACKGROUND OF THE INVENTION

1. Field of the Invention

This invention relates to a device and method for delivering heat to a predetermined surface, and more particularly, to a device and method having features that increase the heat delivered from the device to a predetermined surface of a human body when falling temperatures require more heat to be generated by the device.

2. Background of the Prior Art

A myriad of prior art devices for warming portions of the human body have been patented. These devices are designed to allow the user of the device to position the heating element portions of the respective device upon an area of the users anatomy exposed to relatively cold temperatures. The problem with prior art devices is that the devices do not provide a relatively quick and inexpensive procedure to increase the heat generated by the prior art heating device to counteract a dropping ambient temperature encountered by the user's body portion requiring a corresponding heat increase.

### SUMMARY OF THE INVENTION

It is an object of the present invention to overcome many of the disadvantages associated with prior art heating devices for the human body. A principal object of the present invention is to provide a relatively inexpensive body warming device that is capable of being quickly and inexpensively modified to increase the heat output generated by the body warming device. A feature of the device is a pouch having elastic side portions that enable outer and inner surfaces of the pouch to be separated a distance to receive one or more heating elements that generate a quantity of heat required to warm a predetermined body portion exposed to a known ambient temperature. Another feature of the elastic side portions of the pouch is that the side portions cooperate with the pouch and pouch cover to minimize the volume of the pouch to prevent the heating elements from moving inside the pouch when the device is in motion. An advantage of the device is that it allows the quantity of heating elements to quickly and inexpensively increase when temperatures are dropping. Another advantage of the device is that it allows the quantity of heating elements inside the pouch to be quickly and inexpensively decreased when temperatures are rising. Still another advantage of the device is that it minimizes the pouch volume such that the pouch volume is slightly greater than the volume of the heating elements therein, thereby minimizing movement of the heating elements inside the pouch when the device is in motion, resulting in the retention of the heating elements inside the pouch irrespective of pouch movement and/or pouch orientation upon the human body.

Briefly, the invention provides a device for heating a predetermined surface area of a human body including an expandable pouch for heating a predetermined surface area of a human body; at least one heating element for heating the predetermined surface area of the human body to a predetermined temperature; and means for securing the expandable pouch to the predetermined surface area of the human body, whereby, the pouch, with at least one heating element inserted therein, is secured upon the predetermined surface area of the human body to ultimately heat the predetermined surface area to a predetermined temperature.

The invention further provides a method for heating a predetermined surface area of a human body, the method includes the steps of:

providing an expandable pouch for heating a predetermined surface area of a human body;

providing at least one heating element for heating the predetermined surface area of the human body to a predetermined temperature; and

securing the pouch to the predetermined surface area of the human body, whereby, the expandable pouch, with at least one heating element inserted therein, is secured upon the predetermined surface area of the human body to ultimately heat the predetermined surface area to a predetermined temperature.

The invention also provides a heating device that includes a pouch for heating a predetermined surface area, the pouch including a cover;

at least one heating element for heating the predetermined surface area, the at least one heating element being retained inside the pouch irrespective of the orientation of the heating device;

an outer material secured to an outer surface of the pouch and an inner material secured to an inner surface of the cover, such that the cover is ultimately secured to the outer surface of the pouch to maintain the heating element inside the pouch irrespective of the orientation of the heating device, the outer and inner materials adjustably determining the volume of the pouch such that the pouch can removably receive heating elements varying in size and heating capacity, resulting in a pouch volume slightly larger than the volume of the heating elements, thereby minimizing movement of the heating elements relative to the pouch and preventing the heating elements from escaping from the pouch, irrespective of the movement of the device; and

means for securing the pouch to the predetermined surface area, whereby, the pouch, with at least one heating element inserted therein, is secured upon the predetermined surface area to ultimately heat the predetermined surface area to a predetermined temperature.

### BRIEF DESCRIPTION OF THE DRAWINGS

The objects, advantages and novel features of the present invention, as well as details of an illustrative embodiment thereof, will be more fully understood from the following detailed description and attached drawings, wherein:

FIG. 1 is a top-front, closed position, perspective view of a device for heating a person's wrist in accordance with the present invention.

FIG. 2 is a front elevation view of the device of FIG. 1.

FIG. 3 is the front elevation of FIG. 2, but with the device in an open position.

FIG. 4 is a back elevation view of a device for heating a person's neck in accordance with the present invention.

FIG. 5 is a front elevation view of the device of FIG. 5 in a closed position.

FIG. 6 is the front elevation view of FIG. 5, but with the device in an open position.

FIG. 7 is a back elevation view of an device for heating a person's back in accordance with the present invention.

FIG. 8 is a front elevation view of the device of FIG. 7 in a closed position.

FIG. 9 is the front elevation view of FIG. 8, but with the device in an open position.

### DESCRIPTION OF THE PREFERRED EMBODIMENT

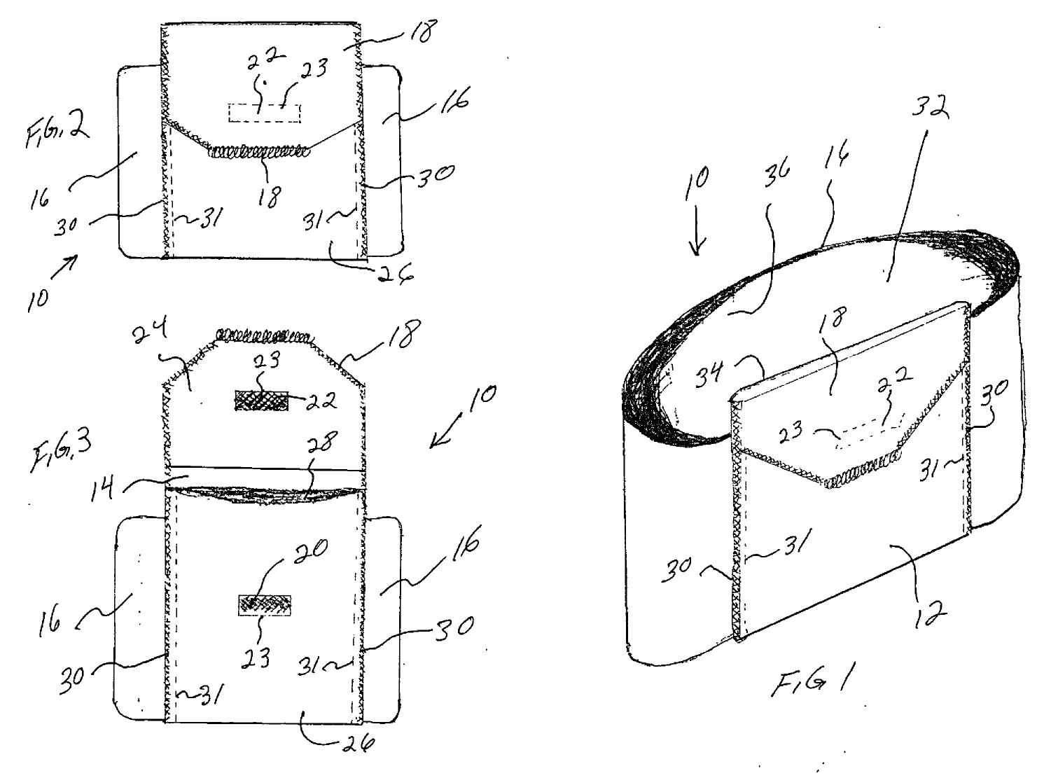

Referring now to the figures, and in particular to FIGS. 1-3, a device for heating a predetermined surface area of a human body, and more specifically, a device for heating a selected portion of a person's wrist and/or hand in accordance with the present invention is denoted by numeral 10. Although the device 10 has a multitude of uses for heating different portions of a person's body, including but not limited to feet, ankles, and forearms, the preferred use for the device 10 is to heat a portion of a person's wrist.

The wrist heating device 10 includes a pouch 12 for heating a predetermined surface area of a person's wrist (not depicted); at least one heating element 14 specified to heat the predetermined surface area of the person's wrist to a predetermined temperature; and a band or strap 16 for securing the pouch 12 to the predetermined surface area of the person's wrist, whereby, the pouch 12 having at least one heating element 14 inserted therein is secured upon the predetermined surface area of the person's wrist to ultimately heat the predetermined surface area to a predetermined temperature. Although one heating element 14 is generally sufficient to comfortably heat a person's wrist, in relatively cold temperatures, the pouch 12 is sized and configured to expand to receive two or more heating elements to generate sufficient heat to maintain a comfortable temperature upon the person's wrist.

The pouch 12 can be manufactured from a myriad of materials that are flexible and that conduct heat, including but not limited to sweat absorbing material such as that used for wristbands, a flannel material, polyesters, a relatively thin, durable animal hide material such as suede, and an elastic material. The pouch 12 includes a substantially rectangular or square configuration having dimensions that correspond to a range of wrist sizes. Generally, four different pouches 12 are configured and dimensioned to correspond to four different wrist sizes characterized as small, medium, large and extra large.

The heating element 14 includes a myriad of heating elements 14 that are commercially available and that insert into the pouch 12. A preferred heating element 14 is manufactured by HeatMax Inc. in Dalton, Ga. 30722. The HeatMax heating element 14 manufactured under the brand name of “Hothands” and identified as item HH2. The Hothands heating element 14 is foldable to promote the insertion into the pouch 12 without damaging the heating element 14 or interfering with the heating parameters of the heating element 14, thereby allowing one or more of the Hothands heating element 14 into the pouch to effectively heat a person's wrist area, irrespective of ambient temperatures.

The pouch 12 includes a cover 18 for retaining the heating element 14 inside the pouch 12, irrespective of the orientation of the heating device 10. The cover 18 is maintained in a closed position by a relatively small portion of a hook or outer material 20 detachably receiving a relatively small portion of a loop or inner material 22. The hook and loop material 20 and 22 is well known to those of ordinary skill as being manufactured pursuant to the trademark “Velcro.” The loop material 22 is secured to an inner surface 24 of the cover 12 via thread 23, staples or similar securing elements, and is ultimately urged to engage the hook material 20, which is secured to an outer surface 26 of the pouch 12, thereby securing the cover 18 to the outer surface 26 of the pouch 12 to maintain the heating element 14 inside the pouch 12, irrespective of the orientation or motion of the heating device 10. The portions of hook and loop material 20 and 22 are dimensioned to allow the cover 18 to be secured to the pouch 12 at multiple positions to promote the expansion or contraction of the volume of the pouch 12 to correspond to the volume of the quantity and dimensions of heating elements 14 ultimately disposed in the pouch 12. The feature of providing a means for varying the attached position of the cover 18 upon the pouch 12 enables the user of the device 10 to maintain the volume of the pouch 12 slightly larger than the volume of the heating elements 14, thereby minimizing movement of the heating elements 14 relative to the pouch 12 and preventing the heating elements 14 from escaping from the pouch 12, irrespective of the movement and orientation of the person wearing the device 10.

The cover 18 is manufactured from the same flexible material as the pouch 12. The cover 18 is dimensioned to cooperate with the portions of hook and loop material 20 and 22 dimensions to allow the cover 18 to be secured to the pouch 12 when the heating element 14 is dimensioned such that the heating element 14 extends beyond a top edge 28 of the outer surface 26 of the pouch 12. The cooperation between the pouch 12, cover 18, and portions of hook and loop materials 20 and 22, enable the pouch 12 and cover 18 to removably receive one of a range of heating elements 14 varying in size and heating capacity; 2.) removably receive multiple heating elements 14; and 3.) removably receive multiple heating elements 14 varying in size and heating capacity to heat a person's hand and/or wrist portion via an inner surface 34 of the pouch 12 engaging a predetermined portion of the person's wrist and/or hand.

The band or strap 16 is fabricated from a stretchable, elastic material and is secured to opposite side portions 30 of the pouch 12 via thread stitching 31, such that a wrist receiving aperture 32 is formed to allow a person's hand to insert between an inner surface 34 of the pouch 12 and an inner surface 36 of the strap 16, until the pouch 12 and strap 16 is snugly secured to the person's wrist, thereby maintaining the pouch 12 with a heating element 14 secured therein by the cover 18 upon a predetermined surface area of the person's wrist to provide sufficient heat to the person's wrist and hand, irrespective of the ambient temperature surrounding the person.

The side portions 30 are fabricated from an elastic or stretchable material well known to those of ordinary skill in the art. The side portions 30 in an “un-stretched” position separate the outer and inner surfaces 26 and 34 substantially about one-half inch, but shorter or greater distances of separation can be specified to cooperate with a selected heating element 14 to be inserted into the pouch 12 that has a relatively “thin” or “thick” dimension. Further, the side portions 30 in a “stretched” position separate the outer and inner surfaces 26 and 34 substantially about one inch to allow the pouch 12 to receive multiple heating elements 14 arranged similarly to a deck of cards when inserted into the pouch 12. The one inch distance of separation can be shorter or greater depending on the number of heating elements 14 that will be ultimately inserted into the pouch 12 to achieve a predetermined heating quantity for the person's wrists when exposed to relatively cold temperatures. The hook and loop materials 20 and 22 are dimensioned to secure the cover 18 upon the pouch 12 irrespective of the distance separating the outer and inner surfaces 26 and 34 of the pouch 12.

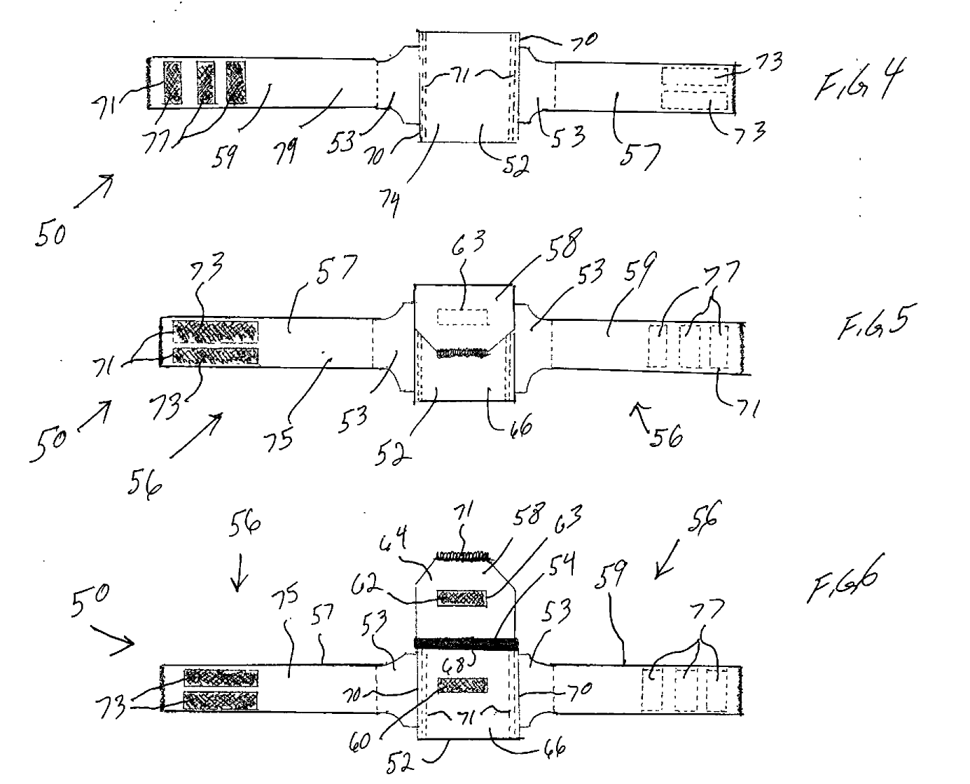

Referring now to the FIGS. 4-6, a device for heating a predetermined surface area of a human body, and more specifically, a device for heating a portion of a person's neck in accordance with the present invention is denoted by numeral 50. Although the device 50 has a multitude of uses for heating different portions of a person's body, including but not limited to lower and upper leg portions, and upper arm portions, the preferred use for the device 50 is to heat a portion of a person's neck.

The neck heating device 50 includes a pouch 52 for heating a predetermined surface area portion of a person's neck (not depicted). The neck device 50 is configured substantially the same as the wrist device 10, but the dimensions for the neck device 50 are relatively larger than corresponding dimensions for the wrist device 10 The neck heating device 50 further includes at least one heating element 54 dimensioned to heat the predetermined surface area of the person's neck to a predetermined temperature; and a band or strap 56 for securing the pouch 52 to the predetermined surface area of the person's neck, whereby, the pouch 52 having at least one heating element 54 inserted therein is secured upon the predetermined surface area of the person's neck to ultimately heat the predetermined surface area to a predetermined temperature. Although one heating element 54 is generally sufficient to comfortably heat a predetermined neck portion, in relatively cold temperatures, the pouch 52 is sized and configured to expand to receive two or more heating elements 54 to generate sufficient heat to maintain a comfortable temperature upon the selected neck portion.

The pouch 52 can be manufactured from a myriad of materials that are flexible and that conduct heat, including but not limited to sweat absorbing material such as that used for wristband 10 described above. The pouch 52 includes a substantially rectangular or square configuration having dimensions that correspond to a range of neck sizes. Generally, four different pouches 52 are configured and dimensioned to correspond to four different neck sizes characterized as small, medium, large and extra large.

The heating element 54 includes the same myriad of wrist heating elements 14 described above that are commercially available and that insert into the wrist heating pouch 12. The same preferred heating element 54 for the neck pouch 52 is manufactured by HeatMax Inc. in Dalton, Ga. 30722. The HeatMax heating element 54 manufactured under the brand name of “Hothands” and identified as item HH2. The Hothands heating element 54 is foldable to promote the insertion into the pouch 52 without damaging the heating element 54 or interfering with the heating parameters of the heating element 54, thereby allowing one or more of the Hothands heating element 54 into the pouch to effectively heat a person's neck area, irrespective of ambient temperatures.

The pouch 52 includes a cover 58 for retaining the heating element 54 inside the pouch 52, irrespective of the orientation of the neck heating device 50. The cover 58 is maintained in a closed position by a relatively small portion of a hook or outer material 60 detachably receiving a relatively small portion of a loop or inner material 62. The hook and loop material 60 and 62 is well known to those of ordinary skill as being manufactured pursuant to the trademark “Velcro.” The loop material 62 is secured to an inner surface 64 of the cover 58 via thread 63, staples or similar securing elements, and is ultimately urged to engage the hook material 60, which is secured to an outer surface 66 of the pouch 52, thereby securing the cover 58 to the outer surface 66 of the pouch 52 to maintain the heating element 54 inside the pouch 52, irrespective of the orientation or motion of the heating device 50. The portions of hook and loop material 60 and 62 are dimensioned to allow the cover 58 to be secured to the pouch 52 at multiple positions to promote the expansion or contraction of the volume of the pouch 52 to correspond to the volume of the quantity and dimensions of heating elements 54 ultimately disposed in the pouch 52. The feature of providing a means for varying the attached position of the cover 58 upon the pouch 52 enables the user of the device 50 to maintain the volume of the pouch 52 slightly larger than the volume of the heating elements 54, thereby minimizing movement of the heating elements 54 relative to the pouch 52 and preventing the heating elements 54 from escaping from the pouch 52, irrespective of the movement and orientation of the person wearing the device 50.

The cover 58 is manufactured from the same flexible material as the pouch 52. The cover 58 is dimensioned to cooperate with the portions of hook and loop material 60 and 62 dimensions to allow the cover 58 to be secured to the pouch 52 when the heating element 54 is dimensioned such that the heating element 54 extends beyond a top edge 68 of the outer surface 66 of the pouch 52. The cooperation between the pouch 52, cover 58, and portions of hook and loop materials 60 and 62, enable the pouch 52 and cover 58 to 1.) removably receive one of a range of heating elements 54 varying in size and heating capacity; 2.) removably receive multiple heating elements 54; and 3.), removably receive multiple heating elements 54 varying in size and heating capacity to heat a person's neck via an inner surface 74 of the pouch 52 engaging a predetermined portion of the person's neck.

The band or strap 56 includes hook and loop strap portions 57 and 59, both fabricated from a stretchable, elastic material and each is secured to respective, opposite side portions 70 of the pouch 52 via thread stitching 71. The hook and loop strap portions 57 and 59 both include enlarged end portions 53 that are secured to and dimensionally correspond to opposite side portions 70 of the pouch 52 to increase a thread 71 length that joins the corresponding strap portions 57 and 59 to the pouch 52, thereby increasing the force binding the strap 56 to the pouch 52. The hook strap portion 57 of the strap 56 includes one or more portions of hook material 73 longitudinally secured via thread 71 to an outer surface 75 of the hook strap portion 57. The loop strap portion 59 of the strap 56 includes one or more portions of loop material 77 laterally secured via thread 71 to an inner surface 79 of the loop strap portion 59. The hook and loop strap portions 57 and 59, and the portions of hook and loop material 73 and 77 cooperate to enable a person to adjustably secure the pouch 52 comfortably about a predetermined portion of the person's neck such that the pouch 52, with a heating element 54 secured therein, is maintained upon a predetermined surface area of the person's neck to provide sufficient heat to the person's neck, irrespective of the ambient temperature surrounding the person.

The side portions 70 are fabricated from an elastic or stretchable material well known to those of ordinary skill in the art. The side portions 70 in an “un-stretched” position separate the outer and inner surfaces 66 and 74 substantially about one-half inch, but shorter or greater distances of separation can be specified to cooperate with a selected heating element 54 to be inserted into the pouch 52 that has a relatively “thin” or “thick” dimension. Further, the side portions 70 in a “stretched” position separate the outer and inner surfaces 66 and 74 substantially about one inch to allow the pouch 52 to receive multiple heating elements 54 arranged similarly to a deck of cards when inserted into the pouch 52. The one inch distance of separation can be shorter or greater depending on the number of heating elements 54 that will be ultimately inserted into the pouch 52 to achieve a predetermined heating quantity for the person's wrists when exposed to relatively cold temperatures. The hook and loop materials 60 and 62 are dimensioned to secure the cover 58 upon the pouch 52 irrespective of the distance separating the outer and inner surfaces 66 and 74 of the pouch 52.

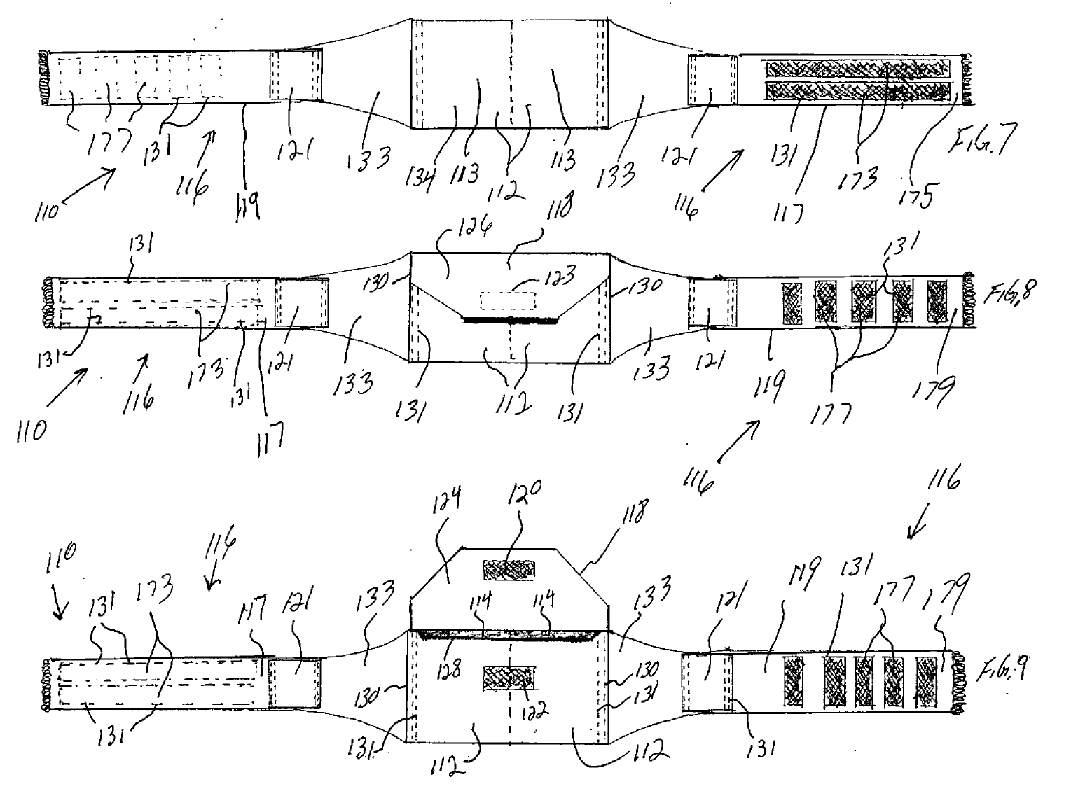

Referring now to the FIGS. 7-9, a device for heating a predetermined surface area of a human body, and more specifically, a device for heating a portion of a person's back and/or chest in accordance with the present invention is denoted by numeral 110. The preferred use for the device 110 is for a person's back, however, the device 110 can be used to heat portions of a person's chest and/or stomach. The back heating device 110 includes a pouch 112 for heating a predetermined surface area of a person's back (not depicted); at least one heating element 114 dimensioned to heat the predetermined surface area of the person's back to a predetermined temperature; and a band or strap 116 for securing the pouch 112 to the predetermined surface area of the person's back, whereby, the pouch 112 having at least one heating element 114 inserted therein is secured upon the predetermined surface area of the person's back to ultimately heat the predetermined surface area to a predetermined temperature.

The pouch 112 can be manufactured from a myriad of materials that are flexible and that conduct heat, including but not limited to sweat absorbing material such as that used for wristband 10 described above. The pouch 112 includes two substantially identical rectangular or square configured portions 113 separated by threaded material fabricated from nylon or similar moisture resistant material. The pouch 112 includes a range of sizes that correspond to the back size of the user. The dimensions for each portion 113 of the pouch 112 are relatively larger than comparable dimensions for the wrist and neck pouches 10 and 50. Generally, four different pouches 112 are configured and dimensioned to correspond to four different back sizes characterized as small, medium, large and extra large.

The heating element 114 includes the same myriad of wrist heating elements 14 described above that are commercially available and that insert into the wrist heating pouch 12. The same preferred heating element 114 for the back pouch 112 is manufactured by HeatMax Inc. in Dalton, Ga. 30722. The HeatMax heating element 114 manufactured under the brand name of “Hothands” and identified as item HH2. The Hothands heating element 114 is foldable to promote the insertion into the pouch 112 without damaging the heating element 114 or interfering with the heating parameters of the heating element 114, thereby allowing one or more of the Hothands heating element 114 into the pouch to effectively heat a person's back area, irrespective of ambient temperatures.

The pouch 112 includes one cover 118 for retaining the heating element 114 inside both portions 113 of the pouch 112, irrespective of the orientation of the back heating device 110. The cover 118 is maintained in a closed position by a relatively small portion of a hook or outer material 120 detachably receiving a relatively small portion of a loop or inner material 122. The hook and loop material 120 and 122 is well known to those of ordinary skill as being manufactured pursuant to the trademark “Velcro.” The loop material 122 is secured to an inner surface 124 of the cover 112 via polymer thread 123, staples or similar securing elements, and is ultimately urged to engage the hook material 120, which is secured to an outer surface 66 of the pouch 112, thereby securing the cover 118 to the outer surface 126 of the pouch 112 to maintain the heating elements 114 inside each respective portion 113 of the pouch 112, irrespective of the orientation or motion of the heating device 110. The portions of hook and loop material 120 and 122 are dimensioned to allow the cover 118 to be secured to the pouch 112 at multiple positions to promote the expansion or contraction of the volume of the pouch 112 to correspond to the volume of the quantity and dimensions of heating elements 114 ultimately disposed in the pouch 112. The feature of providing a means for varying the attached position of the cover 118 upon the pouch 112 enables the user of the device 110 to maintain the volume of the pouch 112 slightly larger than the volume of the heating elements 114, thereby minimizing movement of the heating elements 114 relative to the pouch 112 and preventing the heating elements 114 from escaping from the pouch 112, irrespective of the movement and orientation of the person wearing the device 110.

The cover 118 is manufactured from the same flexible material as the pouch 112. The cover 118 is dimensioned to cooperate with the portions of hook and loop material 120 and 122 dimensions to allow the cover 118 to be secured to the pouch 112 when one or more heating element 114 are dimensioned such that the heating elements 114 extend beyond a top edge 128 of the outer surface 126 of the pouch 112. The cooperation between the pouch 112, cover 118, and portions of hook and loop materials 120 and 122, enable each portion 113 of the pouch 112 and cover 118 to 1.) removably receive one of a range of heating elements 114 varying in size and heating capacity; 2.) removably receive multiple heating elements 114; and 3.) removably receive multiple heating elements 114 varying in size and heating capacity to heat a person's back via an inner surface 134 of the pouch 112 engaging a predetermined portion of the person's back.

The band or strap 116 includes hook and loop strap portions 117 and 119 that are fabricated from the same material as the pouch 112 except for a stretchable, elastic material mid-portion 121 of the strap portions 117 and 119 that enables the person to snugly secure the back heating device 110 about his or her abdomen region. The non-stretchable material forming the strap portions 117 and 119, and pouch 112 is relatively comfortable when engaging a person's skin, thereby enabling the strap 116 and pouch 112 to directly engage the person's skin and maximize heat transfer from the heating element 114 and the predetermined surface area of the person's back. The hook and loop strap portions 117 and 119 both include enlarged end portions 133 that are secured to and dimensionally correspond to opposite side portions 130 of the pouch 112 to increase a thread 131 length that joins the corresponding strap portions 117 and 119 to the pouch 112, thereby increasing the force binding the strap 116 to the pouch 112.

The side portions 130 are fabricated from an elastic or stretchable material well known to those of ordinary skill in the art. The side portions 130 in an “un-stretched” position separate the outer and inner surfaces 126 and 134 substantially about one-half inch, but shorter or greater distances of separation can be specified to cooperate with a selected heating element 114 to be inserted into the pouch 112 that has a relatively “thin” or “thick” dimension. Further, the side portions 130 in a “stretched” position separate the outer and inner surfaces 126 and 134 substantially about one inch to allow the pouch 112 to receive multiple heating elements 114 arranged similarly to a deck of cards when inserted into the pouch 112. The one inch distance of separation can be shorter or greater depending on the number of heating elements 114 that will be ultimately inserted into the pouch 112 to achieve a predetermined heating quantity for the person's wrists when exposed to relatively cold temperatures. The hook and loop materials 120 and 122 are dimensioned to secure the cover 118 upon the pouch 112 irrespective of the distance separating the outer and inner surfaces 126 and 134 of the pouch 112.

The hook strap portion 117 of the strap 116 includes one or more portions of hook material 173 longitudinally secured via thread 131 to an inner surface 175 of the hook strap portion 117. The loop strap portion 119 of the strap 116 includes one or more portions of loop material 177 laterally secured via thread 131 to an outer surface 179 of the loop strap portion 119. The hook and loop strap portions 117 and 119, and the portions of hook and loop material 173 and 177 cooperate to enable a person to adjustably secure the pouch 112 comfortably about a predetermined portion of the person's abdomen such that the pouch 112, with a heating element 114 secured therein, is maintained upon a predetermined surface area of the person's back to provide sufficient heat to the person's back, irrespective of the ambient temperature surrounding the person.

The present invention not only provides the elements to construct a device for heating any portion of a person's body, the present invention also provides a method for heating a predetermined surface area of any portion of a person's body. The method includes the steps of:

providing a pouch for heating a predetermined surface area of a human body;

providing at least one heating element for insertion into the pouch and for heating the predetermined surface area of the human body to a predetermined temperature based upon an ambient temperature; and

securing the pouch to the predetermined surface area of the human body, whereby, the pouch with at least one heating element inserted therein, is secured upon the predetermined surface area of the human body to ultimately heat the predetermined surface area to a predetermined temperature.

The sizing of the heating element to achieve a heat output for the respective surface area of the person's body based upon an ambient temperature is well known to those of ordinary skill in the art.

The foregoing description is for purpose of illustration only and is not intended to limit the scope of protection accorded this invention. The scope of protection is to be measured by the following claims, which should be interpreted as broadly as the inventive contribution permits.

1. A device for heating a predetermined surface area of a human body comprising:
an expandable pouch for heating a predetermined surface area of a human body;
at least one heating element for heating the predetermined surface area of the human body to a predetermined temperature; and
means for securing said expandable pouch to the predetermined surface area of the human body, whereby, said pouch, with at least one heating element inserted therein, is secured upon the predetermined surface area of the human body to ultimately heat the predetermined surface area to a predetermined temperature.

2. The heating device of claim 1 wherein said pouch includes two substantially equal portions with each portion receiving at least one heating element.

3. The heating device of claim 1 wherein said pouch includes a cover for retaining said heating element inside said pouch irrespective of the orientation of said heating device.

4. The heating device of claim 3 wherein said pouch includes means for maintaining said cover in a closed position.

5. The heating device of claim 4 wherein said cover maintaining means includes a hook material secured to an outer surface of said pouch and a loop material secured to an inner surface of said cover, such that said cover is ultimately secured to said outer surface of said pouch to maintain said heating element inside said pouch irrespective of the orientation of said heating device.

6. The heating device of claim 3 wherein said cover includes means for adjusting the volume of said pouch such that said pouch can removably receive heating elements varying in size and heating capacity, such that the volume of said pouch is slightly larger than the volume of said heating elements, thereby minimizing movement of said heating elements relative to said pouch and preventing said heating elements from escaping from said pouch, irrespective of the movement and orientation of the person wearing said device.

7. The heating device of claim 6 wherein said cover includes means for adjusting the volume of said pouch such that said pouch can removably receive multiple heating elements.

8. The heating device of claim 7 wherein said cover includes means for adjusting the volume of said pouch such that said pouch can removably receive multiple heating elements varying in size and heating capacity.

9. The heating device of claim 1 wherein said means for securing said pouch to the predetermined surface area of the human body includes a stretchable strap.

10. The heating device of claim 9 wherein said stretchable strap includes first and second portions having means for detachably joining end portions of said first and second portions, thereby snugly securing said pouch with said heating element therein upon the predetermined surface area of the human body.

11. The heating device of claim 10 wherein said detachably joining means includes a hook material on an inner side of an end portion of said first portion of said strap, and a loop material on an outer side of an end portion of said second portion of said strap.

12. The heating device of claim 11 wherein said hook material includes a plurality of longitudinally disposed strips integrally secured to said inner side of said end portion of said first portion of said strap.

13. The heating device of claim 11 wherein said loop material includes a plurality of laterally disposed strips integrally secured to said outer side of said end portion of said second portion of said strap.

14. A method for heating a predetermined surface area of a human body, said method comprising the steps of:
providing an expandable pouch for heating a predetermined surface area of a human body;
providing at least one heating element for heating the predetermined surface area of the human body to a predetermined temperature; and
securing said pouch to the predetermined surface area of the human body, whereby, said expandable pouch, with at least one heating element inserted therein, is secured upon the predetermined surface area of the human body to ultimately heat the predetermined surface area to a predetermined temperature.

15. The heating method of claim 14 wherein the step of providing a pouch includes the step of dimensioning said pouch to correspond to the predetermined surface area of a human body.

16. The heating method of claim 14 wherein said step of providing a pouch includes the step of providing a cover for retaining said heating element inside said pouch irrespective of the orientation of said heating device.

17. The heating method of claim 16 wherein said step of providing a cover includes the step of maintaining said cover in a closed position.

18. The heating method of claim 17 wherein the step of maintaining said cover in a closed position includes the step of securing a hook material to an outer surface of said pouch and securing a loop material to an inner surface of said cover, such that said cover is ultimately secured to said outer surface of said pouch to maintain said heating element inside said pouch irrespective of the orientation of said heating device.

19. A heating device comprising:
a pouch for heating a predetermined surface area, said pouch including a cover;
at least one heating element for heating the predetermined surface area, said at least one heating element being retained inside said pouch irrespective of the orientation of said heating device;
an outer material secured to an outer surface of said pouch and an inner material secured to an inner surface of said cover, such that said cover is ultimately secured to said outer surface of said pouch to maintain said heating element inside said pouch irrespective of the orientation of said heating device, said outer and inner materials adjustably determining the volume of said pouch such that said pouch can removably receive heating elements varying in size and heating capacity, resulting in a pouch volume slightly larger than the volume of said heating elements, thereby minimizing movement of said heating elements relative to said pouch and preventing said heating elements from escaping from said pouch, irrespective of the movement of said device; and
means for securing said pouch to the predetermined surface area, whereby, said pouch with at least one heating element inserted therein is secured upon the predetermined surface area to ultimately heat the predetermined surface area to a predetermined temperature.

20. The device of claim 19 wherein said pouch includes two substantially equal portions with each portion receiving at least one heating element.
